(19) United States
(12) Patent Application Publication
(10) Pub. No.: US 2020/0022787 A1
(43) Pub. Date: Jan. 23, 2020

(54) Zirconium Post

(71) Applicant: Joseph Ruggirello, Macomb, MI (US)

(72) Inventor: Joseph Ruggirello, Macomb, MI (US)

(21) Appl. No.: 16/439,929

(22) Filed: Jun. 13, 2019

Related U.S. Application Data
(60) Provisional application No. 62/700,433, filed on Jul. 19, 2018.

(51) Int. Cl. A61C 8/00; A61C 13/00; A61C 13/08; C01G 25/02; A61K 6/06; B33Y 70/00; B33Y 80/00; C22C 16/00

(52) U.S. Cl. CPC A61C 8/0022; C01P 2002/77; A61C 13/0019; A61C 13/082; A61C 8/0028; A61C 8/0037; C01G 25/02; A61K 6/0618; A61K 6/0681; B33Y 70/00; B33Y 80/00; C22C 16/00; A61C 2008/0046; C01P 2002/54; A61C 13/0004

(57) ABSTRACT

A dental post made from a zirconia compound, wherein said zirconia compound is at least one of a pure zirconia puck, a zirconia stabilized with yttria (yttrium oxide) compound, a cubic zirconia compound, a Partially Stabilized Zirconia (PSZ) compound, a Tetragonal Zirconia Polycrystal (TZP) compound and a Zirconium based bulk metallic glass (BMG).

### CROSS-REFERENCE TO RELATED APPLICATIONS

The subject patent application claims priority to and all the benefits of U.S. Provisional Patent Application No. 62/700,433 which was filed on Jul. 19, 2018, which is herein incorporated by reference in its entirety.

### BACKGROUND

The present invention relates generally to the field of dentistry. More particularly, it concerns a dental post manufactured from zirconia or a composite consisting of zirconia and various other materials

A post is a prosthetic device that is utilized when there is inadequate tooth structure remaining to support a traditional restoration. The post is a small rod shaped implant, usually metal, that is inserted into the voided root space of the tooth's root and protrudes above the gum line. The post replaces missing tooth structure below the gum line and anchors a manufactured crown to be placed where the diseased tooth was previously located.

The post is utilized after the tooth has had a root canal. A root canal is a treatment used to repair and save a tooth that is badly decayed or has become infected. During a root canal procedure, the nerve and pulp are removed and the inside of the tooth is cleaned. Without a root canal treatment, the tissue surrounding the tooth will become infected and abscessed. The term “Root canal” is also used to describe the natural cavity within the center of the tooth. The pulp or pulp chamber is the soft area within the root canal and the tooth's nerve can be found within the root canal.

There has been a misconception that metallic dental posts played a role in reinforcing or strengthening the crown and the surrounding teeth in which the post was placed. Current dental research shows that metal posts offer no reinforcement benefit and, in fact, can actually weaken the area surrounding the root canal void and place the root canal void and the surrounding teeth at a high risk for fracture.

The most common method of crowning a tooth involves using a dental impression of the tooth to be replaced by a dental practitioner. The impression is then sent out to a lab to create a customized crown. The crown can then be placed at a subsequent dental appointment. Alternatively, the dental office may have its own milling machine and create the crown in the dental office and be inserted in the same office visit.

The dental post is placed into a prepared root canal which positions the dental post. The crown is then attached to the post. The post aids in the distribution of any stresses down onto an alveolar bone (also known as the alveolar process), thereby reducing the risk of a coronal fracture. The alveolar bone is the thickened ridge of bone that contains the tooth sockets (dental alveoli) on the jaw bones that hold teeth.).

When a dental practitioner is deciding if a tooth requires a post and crown rather than a conventional crown to be placed on a stub of a tooth, the following can be considered: the presence of an adequate ferrule, which is a ring shape area above the root which will reinforce the crown as it sits on the root; the sufficiency of the length of canal to retain a post; the curvature and overall anatomy of root canal system; and the sufficiency of the root (radicular) dentine thickness for post preparation.

Posts can be made from metal and non-metallic materials. Traditionally metal posts have been used due to the good corrosion resistance and high yield strength. However, in past decades there was a misconception that metallic dental posts played a role in reinforcing or strengthening the teeth in which they were placed.

Non-metallic posts have a lower incidence of root fracture and coronal fracture than their metallic counterparts from their ability to resist high stress without permanent deformation and breaking. Non-metallic posts are also more aesthetic than metal posts as their optical properties are closer to tooth tissue.

Several types of posts have been developed over the years, for example a fiber reinforced resin post is a form of non-metallic posts that include carbon fiber, fiberglass and woven polyethylene ribbon-reinforced composites. These posts are more likely to fail as their fibers can fray. Ceramic posts are made from a brittle material, and these posts can be too strong which can lead to root fracture. It can be difficult and even impossible to remove a ceramic post.

Carbon fiber posts show very little deformation and can absorb and transfer forces similarly to dentine. Dentine is a calcified tissue of the body and, along with enamel, cementum, and pulp, is one of the four major components of teeth. However, these posts can be dark in color which may lead to an unaesthetic result as the post can shine through the tooth. Glass fiber posts are less brittle than ceramic posts and use unidirectional fibers. However, it is difficult to produce this type of material as fiber bundles require infiltration and wetting with resin. This process can often leave voids on the surface of the fibers, leading to a weakened structure.

### BRIEF DESCRIPTION OF THE DRAWINGS

FIG. 1 is a perspective view of a dental post placed in a void of a tooth root deploying a dental post securely in a complementary recess provided in the crown to be seated thereon;

FIG. 2 is a is a perspective view of a dental post embodying the invention;

FIG. 3 illustrates an embodiment wherein the dental post with a first section and an angled second section at a customizable angle to each other;

FIG. 4 illustrates an embodiment wherein the dental post has an attachable first section and an attachable second section permitting the clinician to both select a size and an angular placement of the post with or without a dental post rod.

FIG. 5 illustrates a different view of the post sections of FIG. 4.

### DETAILED DESCRIPTION

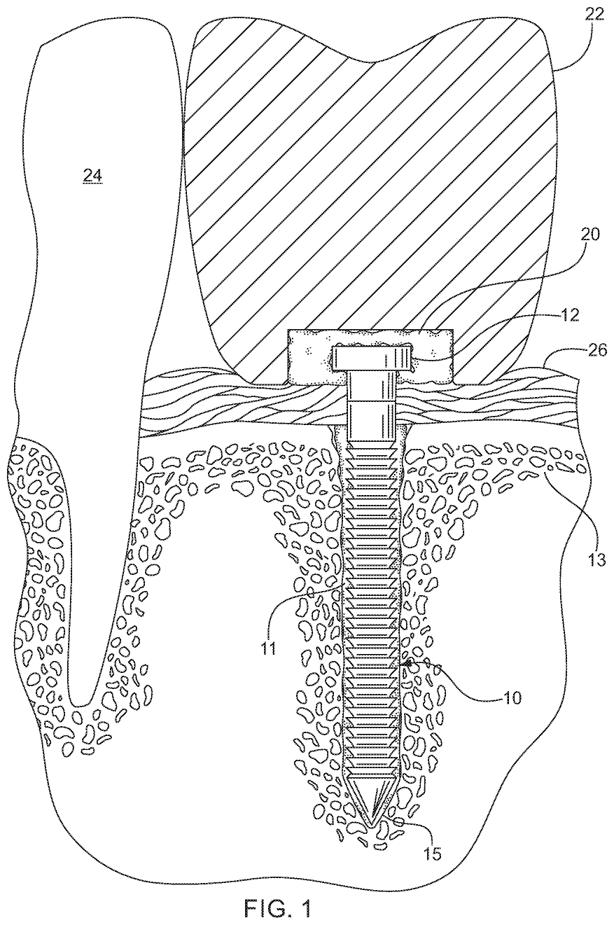

With reference to the Figures, wherein like numerals indicate like parts throughout the several views, FIG. 1 illustrates a crown 22, sometimes known as dental cap, which is a type of dental restoration device. A dental post 10 is placed inside the alveolar bone 13 (also known as the alveolar process). The crowns 22 is typically bonded to the tooth and to the dental post 10 using a dental cement 11.

In an embodiment, the dental post 10 is comprised of a zirconia compound, also known as zirconium which may permit the post 10 to bond to the alveolar bone. The crown can be made of a zirconia stabilized material with yttria (yttrium oxide) can be used as a strong base material for post fabrication. Other compounds with zirconia can include a zirconia stabilized with yttria (yttrium oxide), a Partially Stabilized Zirconia (PSZ) compound, and a Tetragonal Zirconia Polycrystal (TZP) compound. A zirconia dental post 10 will not oxidize or stain the attached crown 22, the surrounding teeth, or any other dental work.

In another embodiment, is a pigment is added to the zirconia compound to match a shade and the esthetic appearance (color and shading) of the mouth and of the surrounding teeth, a bridge or any orthodontic device.

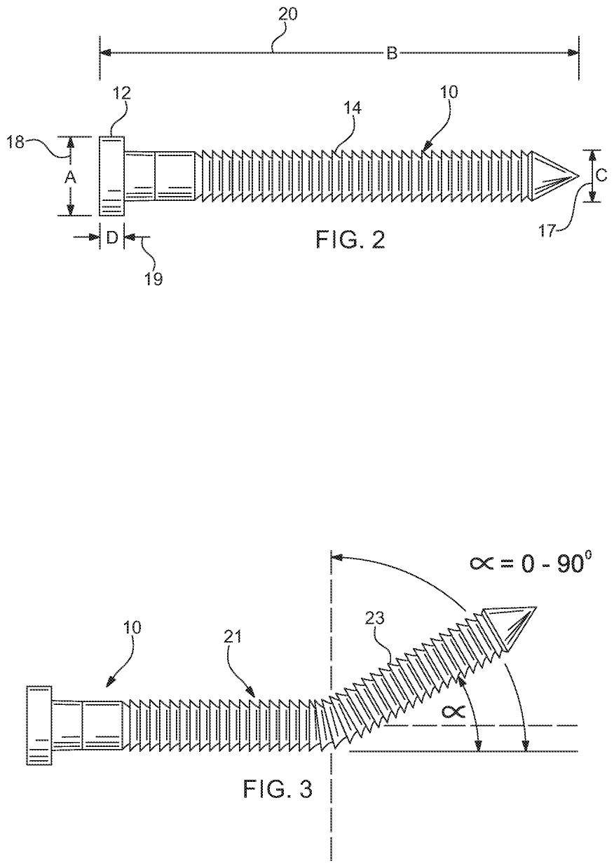

As shown in FIG. 2, the dental post 10 comprises a dental post head 12 abutted to a first end of a dental post shaft 16. The dental post shaft 14 having a taper at a second end 24.

Now with reference to FIG. 3, in another embodiment, the dental post 10 has a total dental post length (dimension B) of 10 mm to 30 mm including the dental post head 12. The dental post head 12 can have a dental post head (dimension A) ranging from 1 mm to 10 mm and a dental post head width (dimension D) ranging 0.5 mm to 5 mm. A dental post shaft 14 can have a dental post shaft diameter (dimension C) ranging from 0.05 mm to 5 mm.

The dental post shaft 14 can have a varying surface textures. The surface texture can be a barbed surface as shown in FIG. 2 or alternatively, the surface texture can have a barbed surface pattern, a ribbed surface pattern, a serrated surface pattern, a cross hatched pattern and a threaded surface pattern.

In an embodiment, the dental post 10 can be made form a zirconia-based bulk metallic glass (BMG) compound, for example a Zr61Ti2Cu25Al12ZT1 alloy compound. This amorphous compound exhibits a good incorporation of high strength, high fracture toughness, and lower Young's modulus. Metallic glasses (or amorphous alloys) as compared to the conventional crystalline metals possess substantially uniform microstructure, with no defects such as dislocation and grain boundary. Also, a short-range arrangement of atoms takes place in the amorphous solids as compared to a long-range order of crystalline solids resulting in many desirable properties such as high yield strength, large elastic strain of approximately 2%, and yet have excellent corrosion resistance.

In addition to BMG, other elements and compounds can be mixed or layered with the zirconia compounds. The other elements and compounds can include a titanium element, a hydroxyapatite (Ti-HA-Ti) compound, a hydroxyapatite (Zr02-HA-Ti) compound, an aluminum oxide compound, and a hydroxyapatite (Al203-HA-Ti) compound, but can be any compound or element which will increase the post 10 strength and ensure an acceptable Young's modulus of deformation.

Now with reference to FIGS. 1 and 3, the dental post 10 comprises a first sub-shaft 21 and a second sub-shaft 23 angularly affixed to the first sub-shaft 21 at a customizable angle. Also, the first sub-shaft 21 has a first sub-shaft length of 1-10 mm and the second sub-shaft 23 has a second sub-shaft length of 1-10 mm. The customizable angle can range from 0 to 90 degrees, permitting the practitioner to place the post into a root canal void 15. The customizable angle, for example, permits a manufacturer of dental post to produce a large quantity of dental post at a common angle of 30 degrees. This feature is especially important when the root canal void 15 is not perpendicular to the alveolar bone 13. In practice, the dental practitioner prefers the dental post head 12 of the dental post 10 perpendicular to the alveolar bone 13 to better affix the crown 22 onto the dental post 10.

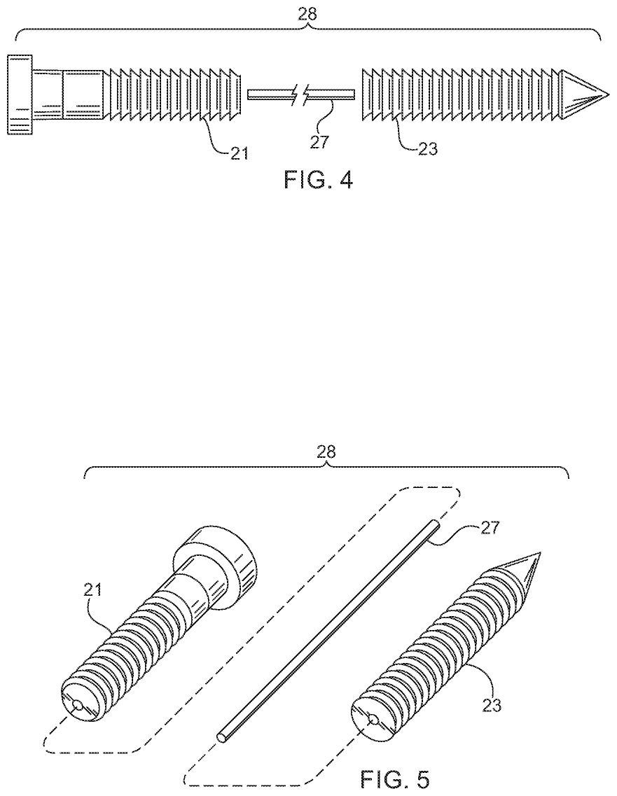

FIG. 4 illustrates an embodiment of the post in which a docking dental post 28 comprises a first sub-shaft 21 and a second sub-shaft 23. The first sub-shaft 21 and the second sub-shaft 23 can be joined together to form the docking dental post 28. The joining can be achieved using a dental cement, as described herein. Additionally, a first proximal end of the first sub-shaft 21 or a second proximal end of the second sub-shaft 23 are malleable and therefore, be filed or machined by the dental practitioner to obtain a customizable angle between the first sub-shaft 21 and the second sub-shaft 23.

In an embodiment, the first sub-shaft 21 and the second sub-shaft 23 each have a shaft bore in which a dental post rod can be inserted. The dental post rod is adjustable, thereby permitting the practitioner to adjust the dental post rod to the customizable angle and join the first sub-shaft 21 and the second sub-shaft 23 with a dental cement.

When affixing the dental post 10 to the crown 22 or even when joining the first sub-shaft 21 and the second sub-shaft 23, a dental cement 11 can used by the dental practitioner. Additionally, to increase the stability of the placed dental post 10, the dental cement 11 can also be placed inside the root canal void 15 to cement the post 10 to the alveolar bone 13. The dental cement 11 can a standard dental affixing compound.

The Dental cement 11 ideally has the following characteristics; it is non-irritating, as many cements are acidic and therefore can be an irritant to the pulp. However, on setting the post 10 or the crown 22, there may be a rapid increase in pH of the mouth. The dental cement must provide a good marginal seal to prevent marginal leakage and be resistant to dissolution in saliva, or in any oral fluid. A primary cause of failure of cements is due to dissolution of the cement at the margins of a restoration. The dental cement should have a high strength in tension, shear and compression to resist stresses at the restoration-tooth interface. It should have adequate working and setting times, appear aesthetically pleasing have good thermal and chemical resistance. The dental cement should radiologically opaque for diagnostic purposes on X-rays and provides good retention. If an adhesive bond can be created between the cement and the restorative material, this can greatly enhance the retention. Otherwise, the retention is governed by the geometry of the tooth preparation.

To better adhere the post 10 to the crown 22 or to have the dental post 10 adhere to the root canal void 15 or join the first sub-shaft 21 and the second sub-shaft 23, a zirconia based dental filling compound can be utilized. In an embodiment, a zirconia dental cement comprises the following material (with respect to the total weight thereof): 45 to 85% of zirconia powder; 14 to 54% of a hydraulic inorganic binder; 0.5% to 0% of maleic acid, citric acid, or tartaric acid as a weakly acidic hardening control agent; and 5% or less of a pozzolan component.

In yet another embodiment, the dental post 10 is treated with an airborne-particle abrasion material, thereby permitting a “rough surface” for better adhesion to the alveolar bone 13.

The dental post 10 can be coated with a silane coupling agent or just be treated without the airborne-particle abrasion material, and only the silane coupling agent. A silane coupling agent can form a durable bond between organic and inorganic materials. Encounters between dissimilar materials often involve at least one member that's siliceous or has surface chemistry with siliceous properties; silicates, aluminates, borates, etc., are the principal components of the earth's crust. Interfaces involving such materials have become a dynamic area of chemistry in which surfaces have been modified to generate desired heterogeneous environments or to incorporate the bulk properties of different phases into a uniform composite structure.

The general formula for a silane coupling agent typically shows the two classes of functionality. X is a hydrolyzable group typically alkoxy, acyloxy, halogen or amine. Following hydrolysis, a reactive silanol group is formed, which can condense with other silanol groups, for example, those on the surface of siliceous fillers, to form siloxane linkages. Stable condensation products are also formed with other oxides such as those of aluminum, zirconium, tin, titanium, and nickel. Less stable bonds are formed with oxides of boron, iron, and carbon. Alkali metal oxides and carbonates do not form stable bonds with Si—O—. The R group is a nonhydrolyzable organic radical that may possess a functionality that imparts desired characteristics. The result of reacting an organosilane with a substrate ranges from altering the wetting or adhesion characteristics of the substrate, utilizing the substrate to catalyze chemical transformations at the heterogeneous interface, ordering the interfacial region, and modifying its partition characteristics. Significantly, the silane coupling includes the ability to affect a covalent bond between organic and inorganic materials.

In yet another embodiment a system deploying a computer and an array of sensors to generate a tomographic map of an inside of a mouth to determine at least one of a tooth placement, a jaw placement, an adequate ferrule (coronal tooth structure), a sufficient length of canal to retain a post, a curvature and overall anatomy of root canal system, and a sufficient root (radicular) dentine thickness for post preparation to determine a schematic of at least one a plethora of problematic areas of said mouth. Then a series of programmatic steps to be executed on a milling machine to produce a dental post based upon said schematic of the map. The milling machine produces the dental post from at least one material from a pure zirconia puck, a zirconia stabilized with yttria (yttrium oxide) compound, a cubic zirconia compound, a Partially Stabilized Zirconia (PSZ) compound, a Tetragonal Zirconia Polycrystal (TZP) compound and a Zirconium bulk metallic glass (BMG).

In another embodiment, the dental post 10 can be produced using a computer numerical controlled (CNC) milling machining, by a micro-injection molding system, or a combination of the two.

In the CNC milling process, the zirconium compound is milled using a CNC milling machine until a preliminary dental post is obtained. The preliminary dental post is then pressed utilizing a hot isostatic pressing (HIP) machine at a high temperature, for example at 1100° C. in an argon atmosphere for a pressing time, for example, one hour. This process is a sintering process which compacts and forms a solid mass of material by heat or pressure without melting it to the point of liquefaction. However, one who is skilled in the art of HIP would be capable to determine when all, if not most of the impurities are removed. HIP is used to form and densify containerized powder shapes and containerless metal, ceramic and plastic parts. With typical pressures from 1,035 to 2,070 bar (15,000 to 30,000 psi) and temperatures up to 2,000° C. (4,000° F.), HIP can achieve 100% of maximum theoretical density and improve the ductility and fatigue resistance of critical, high-performance materials. Then the HIP treated preliminary dental post is milled again by the CNC machine, producing the dental post 10.

The micro-injection molding process is a highly-specialized manufacturing process that produces extremely small, high-precision parts and components with micron tolerances. The process starts in a tooling department where a mold is created that has a cavity in the shape of the dental post 10. Various zirconium compounds or resin are rapidly injected into the cavity, creating the dental post 10. The process stacks the various compounds by injecting a layering material. The layering material can be other zirconium compounds, and elements, for example, a titanium element, a hydroxyapatite (Ti-HA-Ti) compound, a hydroxyapatite (Zr02-HA-Ti) compound, an aluminum oxide compound, and a hydroxyapatite (Al203-HA-Ti) compound can be injected into a micro-injection molding machine to produce a pre-sinter post. The pre-sinter post is then heated and/or pressed to produce a post-sinter post in a hot isostatic pressing machine (HIP) at 1100° C. in an argon atmosphere for one hour producing a post-sinter post. Sintering is the process of compacting and forming a solid mass of material by heat or pressure without melting it to the point of liquefaction. The post-sinter post is the milled, preferably in a CNC milling machine until a dental post 10 is produced.

Computing devices such as those discussed herein generally each include instructions executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, HTML, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media. A file in a computing device is generally a collection of data stored on a computer readable medium, such as a storage medium, a random-access memory, etc.

A computer-readable medium includes any medium that participates in providing data (e.g., instructions), which may be read by a computer. Such a medium may take many forms, including, but not limited to, non-volatile media, volatile media, etc. Non-volatile media include, for example, optical or magnetic disks and other persistent memory. Volatile media include dynamic random access memory (DRAM), which typically constitutes a main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

With regard to the media, processes, systems, methods, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be apparent to those of skill in the art upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

All terms used in the claims are intended to be given their broadest reasonable constructions and their ordinary meanings as understood by those skilled in the art unless an explicit indication to the contrary in made herein. In particular, use of the singular articles such as “a,” “the,” “said,” etc. should be read to recite one or more of the indicated elements unless a claim recites an explicit limitation to the contrary.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. A dental post, comprising a zirconia compound, wherein said zirconia compound is at least one of a pure zirconia puck, a zirconia stabilized with yttria (yttrium oxide) compound, a cubic zirconia compound, a Partially Stabilized Zirconia (PSZ) compound, a Tetragonal Zirconia Polycrystal (TZP) compound and a Zirconium based bulk metallic glass (BMG).

2. The dental post of claim 1, further comprises a dental post head abutted to a first end of a dental post shaft, and the dental post shaft has a taper at a second end of the dental post shaft.

3. The dental post of claim 2, wherein the dental post shaft has a dental post length of 5 mm to 20 mm and a first diameter of 1 mm to 5 mm and a dental post shaft having a diameter of 1 mm to 5 mm.

4. The dental post of claim 1, wherein the dental post has a first surface texture, wherein the first surface texture is at least one of a barbed surface pattern, a ribbed surface pattern, a serrated surface pattern, a cross hatched pattern and a threaded surface pattern.

5. The dental post of claim 1, wherein the zirconium based bulk metallic glass (BMG) is a Zr61Ti2Cu25Al12ZT1 compound.

6. The dental post of claim 1, where wherein a shade of the zirconia compound is selected to match at least one of a crown, a bridge or an orthodontic device.

7. The dental post of claim 1, wherein said post is produced in at least a milling machine, a CNC machine, a 3-D printer and in a mold.

8. The dental post of claim 1, wherein said post is treated with an airborne-particle abrasion material.

9. The dental post of claim 1, wherein said post is coated with a silane coupling agent.

10. The dental post of claim 1, wherein the dental post comprises a first sub-shaft and a second sub-shaft, said second sub-shaft angularly affixed to the first sub-shaft at a customizable angle of 0 to 90 degrees.

11. The dental post of claim 10, wherein the first sub-shaft has a first sub-shaft length of 1-10 mm and the second sub-shaft has a second sub-shaft length of 1-10 mm.

12. The dental post of claim 11, wherein the first sub-shaft and the second sub-shaft are malleable, thereby permitting a practitioner to select a customizable angle between the first sub-shaft and the second sub-shaft.

13. The dental post of claim 12, wherein the first sub-shaft and the second sub-shaft each have a shaft bore; and
a dental post rod.

14. The dental post rod of claim 13, wherein the dental post rod is adjustable, thereby permitting the practitioner to adjust the dental post rod to the customizable angle.

15. A method of producing a dental post comprising:
milling a zirconium compound in a CNC machine until a preliminary dental post is obtained;
pressing said preliminary dental post in a hot isostatic pressing (HIP) machine at a first temperature of 800-2000 Celsius and in an argon atmosphere of pressure is 1,035-2,070 bar for a pressing time; and
remilling the preliminary dental post in the CNC machine until the dental post is produced.

16. The method of claim 15, wherein said zirconium compound is at least one of from a pure zirconia puck, a zirconia stabilized with yttria (yttrium oxide) compound, a cubic zirconia compound, a Partially Stabilized Zirconia (PSZ) compound, a Tetragonal Zirconia Polycrystal (TZP) compound and a Zirconium based bulk metallic glass.

17. A method of producing a dental post comprising:
injecting a zirconium compound into a mold of a micro-injection molding machine;
alternating the injection of a layer of a layering material, said layering material is at least one of a titanium element, a hydroxyapatite (Ti-HA-Ti) compound, a hydroxyapatite (Zr02-HA-Ti) compound, an aluminum oxide compound, said zirconium compound and a hydroxyapatite (Al203-HA-Ti) compound to form a pre-sinter post; and
heating the pre-sinter post in a hot isostatic pressing (HIP) machine at a first temperature of 800-2000 Celsius and in an argon atmosphere of pressure is 1,035-2,070 bar for a pressing time to produce a post-sinter post; and
milling the post-sinter post in a CNC machine until a dental post is produced.

18. The method of claim 17, wherein said zirconium compound is at least one of from a pure zirconia puck, a zirconia stabilized with yttria (yttrium oxide) compound, a cubic zirconia compound, a Partially Stabilized Zirconia (PSZ) compound, a Tetragonal Zirconia Polycrystal (TZP) compound and a Zirconium based bulk metallic glass.

19. The method of claim 17, further comprising:
adjusting a dental post rod to a customizable angle by a practitioner.

20. A dental filling compound to affix a crown to a dental post; said dental filling compound comprising:
45% to 85% of a zirconia powder;
14% to 54 of a hydraulic inorganic binder;
0.5% or less of at least one of a maleic acid, a citric acid, and a tartaric acid as a weakly acidic hardening control agent; and
5% or less of a pozzolan component.
